(19) United States
(12) Plant Patent Application Publication
(10) Pub. No.: US 2017/0034975 P1
(43) Pub. Date: Feb. 2, 2017

(54) Sambucus 'SANIVALK'

(50) ;

(71) Applicant: Peter Brand, Hatfield (GB)

(72) Inventor: Peter Brand, Hatfield (GB)

(73) Assignee: A. Brand and Sons, Hatfield (GB)

(21) Appl. No.: 14/756,095

(22) Filed: Jul. 31, 2015

(51) Int. Cl. A01H 5/00

(52) U.S. Cl. PLT/226

(57) ABSTRACT

A new and distinct Sambucus cultivar named ‘SANIVALK’ is disclosed, characterized by a very hardy plant of compact and bushy growth, dark green leaves with narrow cream-colored margins, young leaves with intense yellow margins, and free flowering. The new variety is a Sambucus normally produced as an outdoor garden or container plant.

### LATIN NAME OF THE GENUS AND SPECIES

Sambucus nigra

### VARIETY DENOMINATION

‘SANIVALK’

### BACKGROUND OF THE INVENTION

The new cultivar is the product of chance discovery. The new variety originated as a naturally occurring branch mutation of an unpatented unnamed proprietary seedling variety of Sambucus nigra.

The new variety was discovered in April of 2013 by the inventor Peter Brand a citizen of England in a nursery in Hatfield, United Kingdom.

Asexual reproduction of the new cultivar was performed by softwood cuttings. This was first performed at a nursery in Hatfield, United Kingdom in Summer of 2013 and has shown that the unique features of this cultivar are stable and reproduced true to type in 3 successive generations.

### SUMMARY OF THE INVENTION

The cultivar ‘SANIVALK’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SANIVALK’ These characteristics in combination distinguish ‘SANIVALK’ as a new and distinct Sambucus cultivar:

1. Relatively compact and bushy growth.

2. Mature leaves fresh dark green with narrow cream-colored margins.

3. Young leaves intense yellow margined.

4. Free flowering.

5. Very hardy.

Plants of the new cultivar ‘SANIVALK’ are similar to plants of the seed parent in most horticultural characteristics, however plants of the new cultivar ‘SANIVALK’ differs in the following;

1. The new variety has dark green leaves margined with cream whereas the parent has dark green leaves that are not variegated.

2. The new variety has young leaves with intense yellow margin, whereas the parent has no margin coloration.

### COMMERCIAL COMPARISON

Plants of the new cultivar ‘SANIVALK’ are comparable to the unpatented commercial variety Sambucus ‘Madonna’. The two Sambucus varieties are similar in most horticultural characteristics; however, the new variety ‘SANIVALK’ differs in the following:

1. Mature leaf color of the new variety is dark green narrow cream-colored margins whereas the comparator has dark green leaves broadly margined in cream-colored to yellow-cream.

Plants of the new cultivar ‘SANIVALK’ can also be comparable to the unpatented commercial variety Sambucus ‘Marginata’. The two Sambucus varieties are similar in most horticultural characteristics; however, the new variety ‘SANIVALK’ differs in the following:

1. Mature leaf color of the new variety is dark green narrow cream-colored margins whereas the comparator has green leaves with cream-white margins.

### BRIEF DESCRIPTION OF THE PHOTOGRAPHS

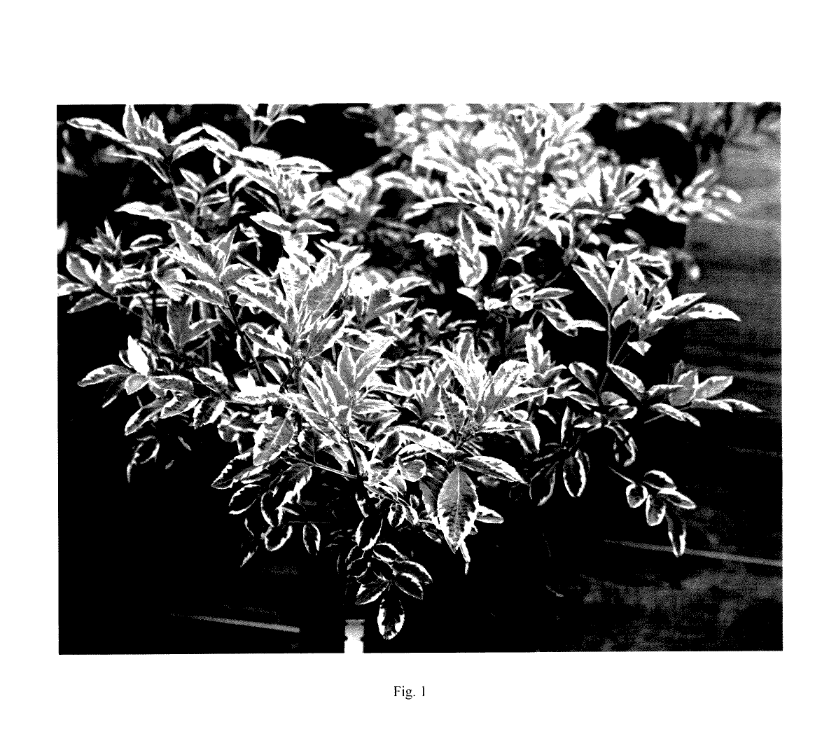

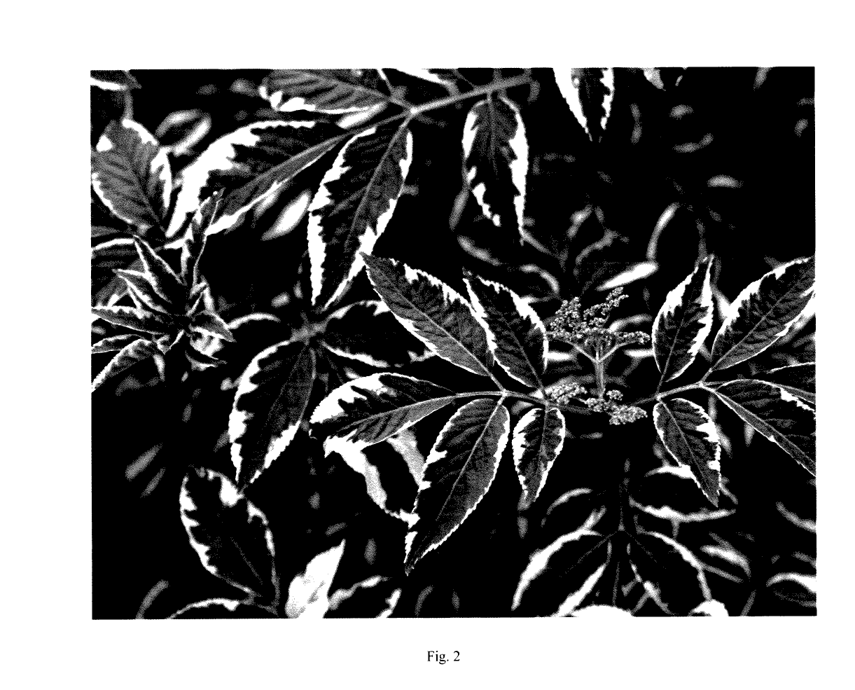

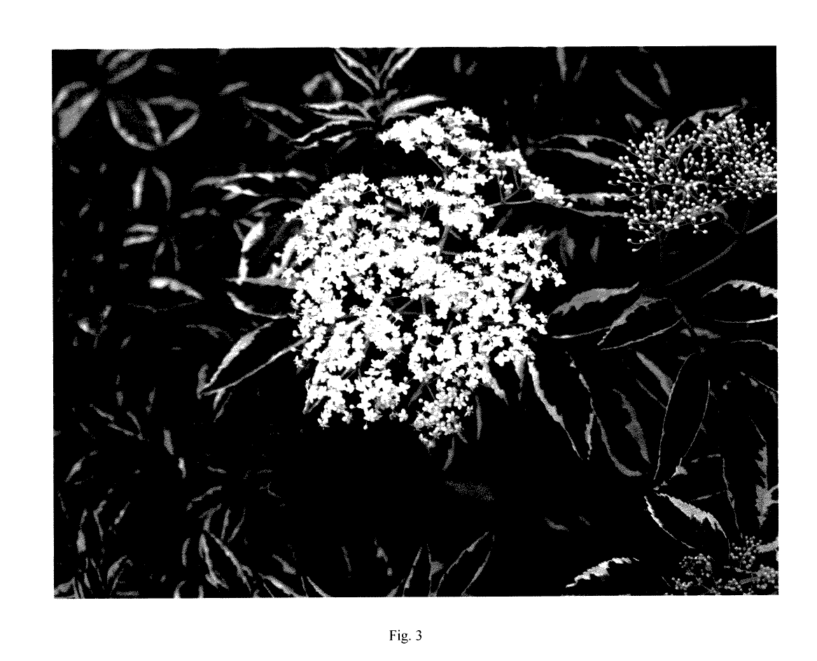

The accompanying photograph in FIG. 1 illustrates in full color a typical plant of ‘SANIVALK’ grown outdoors, in a 3 gallon pot. Age of the plant photographed is approximately 1 year old from a rooted cutting. FIG. 2 illustrates a close up of the foliage. FIG. 3 illustrates flowers and foliage. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 1996 except where general terms of ordinary dictionary significance are used. The following observations and measurements describe ‘SANIVALK’ plants grown in March in a poly greenhouse, in Grand Haven, Mich. USA. The growing temperature ranged from 40° F. to 70° F. during the day and from 30° F. to 45° F. during the night. General light conditions are bright, normal sunlight. Measurements and numerical values represent averages of typical plant types.
Botanical classification: Sambucus nigra ‘SANIVALK’
Propagation:
   Type of propagation typically used.—Softwood cutting.
   Time to initiate roots.—21 days at 20° C. average temperature.
   Time to produce a rooted young plant.—60 days at 15-20° C. average temperature.
   Root description.—Moderately dense, moderately free-branching, medium thickness, non-fibrous, fleshy. Color is white.
Plant:
   Age of plant described.—1 year from rooted cutting.
   Plant type.—Perennial shrub.
   Container size of the plant described.—3 gallon pot.
   Appropriate containers.—1, 2, 3, 5 gallon pots.
   Growth habit.—Upright, mounded.
   Overall plant shape.—Upright, mounded.
   Height.—0.75 m in a #3 container.
   Plant spread.—1 m in a #3 container.
   Growth rate.—Medium fast.
   Crop time to finish.—2¼″ pot to #1 in 4 months.
   Plant vigor.—Good.
   Characteristics of primary lateral branches.—Quantity: 8 Branching habit: Opposite, Pinching required as shearing helps. Branch appearance: Round Length: 0.5 m-3 gallon container. Diameter: 0.7 cm. Color: RHS Yellow-green 145A Pubescence: none Stem aspect/angle: 20-45°Strength: Good Other branch characteristics: Purpling at nodes RHS Red-purple 60A.
   Internode length.—Average 11 cm.
Foliage:
   Leaf.—Quantity of leaflets per leaf: 5 Average Length: 6.5-10 cm Average Width: 3-4.5 cm Shape: Lanceolate Apex: Acute Base: Cuneate Margin: Serrulate Texture of top surface: Smooth Texture of bottom surface: Puberulent Pubescence: Leaf underside Color: Young foliage upper side: Base color RHS Green 143A, variegation Yellow 8A. Young foliage under side: Base color RHS Green 138B, variegation Yellow 3C Mature foliage upper side: Central color RHS Green 137A. Variegation on margins RHS Yellow-orange 14D, Fade to Green 138B. Mature foliage under side: Central color RHS Green 136B. Variegation on margins RHS Yellow 12D, Fade to Green 138B. Venation: Type: Pinnate-netted Venation color upper side: RHS Green 137A. Venation color under side: RHS Green 136B Petiole: Length: 3 cm Diameter: 2 mm Color: Upper side: RHS Green 136B Lower side: RHS Green 136B Texture: Upper side: Canaliculated (sunken central groove) Lower side: Smooth Other traits: Variegated margin approx. 0.5 cm from edge.
Flower:
   Flower arrangement.—Terminal umbel-like cymes.
   Flower shape.—Rotate.
   Natural flowering season.—End April/early May.
   Inflorescence height.—10 cm.
   Inflorescence width.—16 cm.
   Inflorescence and flower type and habit.—Umbel of rotate flowers.
   Flower longevity on plant.—1-2 weeks.
   Persistent or self-cleaning.—Self-cleaning.
   Quantity of flowers per inflorescence.—220.
   Quantity of flowers per plant.—600-700 on a 3 gallon container.
   Bud.—Shape: Spherical Length: 3 mm Diameter: 2 mm Color: RHS Yellow 11C.
   Flower size.—Diameter: 6 mm Length: 4 mm Depth (height): 4 mm.
   Petals.—Appearance: Rounded facing out Arrangement: Single whorl, not fused. Petal length: 3 mm Petal width: 2 mm Quantity: 5 Texture: Smooth Apex: Acute Shape: Obovate Base: Obtuse Margin: Entire, slightly undulate Aspect: Upright.
   Petal color.—When opening: Upper surface: RHS White 155D Lower surface: RHS White 155D Fully opened: Upper surface: RHS White 155D Lower surface: RHS White 155D Fading to Color: Upper and lower: RHS White 155A.
   Sepals.—Quantity per flower: 4 Arrangement: Single flat whorl Shape: Ovate Length: less than 1 mm Width: less than 1 mm Apex: Acute Base: Obutuse Margin: Entire Texture: Upper: Smooth Lower: Smooth Color: Upper Surface: RHS Green 142B Lower Surface: RHS Green 142B.
   Calyx.—Shape: Radial Length: 1 mm Diameter: 1 mm.
   Peduncle.—Length: 8-10 cm Diameter: 3 mm Angle: 45°Strength: Fibrous softwood, very strong. Texture: Smooth Color: RHS Yellow-green 145B.
   Pedicel.—Length: 4-6 mm Diameter: 0.5 mm Color: RHS Yellow-green 145B Angle: 45°Texture: Smooth Strength: Fibrous, strong.
   Fragrance.—Very slight/herbaceous.
Reproductive organs:
   Stamens.—Number: 4 Filament length: 1 mm. Filament color: RHS White 155D.
   Anthers.—Shape: Oblong Length: less than 1 mm Color: RHS yellow 8A Pollen: Color: RHS White 155D Quantity: Lots.
   Pistil.—Number: 1 Length: 0.5 mm Style: Length: less than 1 mm Color: RHS White 155D Stigma: Shape: Truncate Color: RHS White 155D. Ovary Color: RHS White 155D.
Other characteristics:
   Seeds and fruits.—Not observed to date.
   Disease/pest resistance.—Highly tolerant to pests and diseases.
   Lowest temperature tolerance.—At least to USDA zone 4.
   Highest temperature tolerance.—At least to USDA zone 9.
   Garden performance.—Excellent garden performance; very tolerant to rain and wind.

What is claimed is:

1. A new and distinct cultivar of Sambucus plant named ‘SANIVALK’ as herein illustrated and described.
